(19) United States
(12) Patent Application Publication
(10) Pub. No.: US 2026/0053334 A1
(43) Pub. Date: Feb. 26, 2026

(54) SYSTEMS AND METHODS FOR MONITORING SCANNING ACCURACY

(71) Applicant: Align Technology, Inc., San Jose, CA (US)

(72) Inventor: Ofer Saphier, Rehovot (IL)

(21) Appl. No.: 19/304,975

(22) Filed: Aug. 20, 2025

Related U.S. Application Data
(60) Provisional application No. 63/685,542, filed on Aug. 21, 2024.

(51) Int. Cl. A61B 1/00; A61B 1/24; A61C 9/00

(52) U.S. Cl. CPC A61B 1/00057; A61B 1/00009; A61B 1/24; A61C 9/006

(57) ABSTRACT

Systems and methods for monitoring scanning accuracy are provided. In some embodiments, a method for monitoring accuracy of an intraoral scanner includes receiving results of at least one intraoral scanning session. The at least one intraoral scanning session can involve obtaining scan data of an intraoral structure from a scanner and combining the scan data to generate a 3D digital representation of the intraoral structure. The method can include evaluating scanning accuracy of the scanner based on the results of the at least one intraoral scanning session, and outputting an indication of the scanning accuracy on a display.

### CROSS-REFERENCE TO RELATED APPLICATION(S)

The present application claims the benefit of priority to U.S. Provisional Patent Application No. 63/685,542, filed Aug. 21, 2024, which is incorporated by reference herein in its entirety.

### TECHNICAL FIELD

The present technology generally relates to scanning technology, and in particular, to systems and methods for monitoring scanning accuracy.

### BACKGROUND

Scanning may be used to obtain 3D information of anatomical structures of a patient for diagnostic and therapeutic purposes. For example, intraoral scans of a patient's dental arch may be used to diagnose the patient with a dental condition, develop a treatment plan for the dental condition (e.g., administration of dental appliances, installation of dental prosthetics), and/or to monitor treatment progress. The accuracy and efficacy of diagnosis and treatment may be compromised if there are errors in the scan data. In some instances, such errors may be attributable to poor scanning accuracy, which may arise due to hardware or software malfunction, calibration drift, and/or improper scanning protocols, for example. However, conventional scanner systems generally lack the capability to monitor scanning accuracy and to determine the causes of accuracy issues.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A schematically illustrates a system for performing intraoral scanning and/or generating 3D digital representations of a patient's intraoral cavity, in accordance with embodiments of the present technology.

FIG. 1B is a partially schematic illustration of an example scanner that may be used in the system of FIG. 1A, in accordance with embodiments of the present technology.

FIG. 2 is a flow diagram illustrating a method for monitoring scanning accuracy, in accordance with embodiments of the present technology.

FIG. 3 is a flow diagram illustrating a method for evaluating scanning accuracy based on stitching errors, in accordance with embodiments of the present technology.

FIG. 4 illustrates an example of a scanning trajectory for detecting stitching errors, in accordance with embodiments of the present technology.

FIG. 5 is a flow diagram illustrating a method for evaluating scanning accuracy based on bite penetration, in accordance with embodiments of the present technology.

FIG. 6 illustrates a 3D digital representation of a patient's dental arches with bite penetration, in accordance with embodiments of the present technology.

FIG. 7 is a flow diagram illustrating a method for evaluating scanning accuracy based on comparisons of different scan data, in accordance with embodiments of the present technology.

FIG. 8A schematically illustrates scan data divided into a set of first scans and a set of second scans, in accordance with embodiments of the present technology.

FIG. 8B schematically illustrates scan data including a selected subset of scans, in accordance with embodiments of the present technology.

### DETAILED DESCRIPTION

The present technology relates to scanning of anatomical structures, such as intraoral scanning of a patient's dental arch. In some embodiments, for example, a method for monitoring accuracy of an intraoral scanner includes receiving results of at least one intraoral scanning session. The at least one intraoral scanning session can involve obtaining scan data of an intraoral structure from a scanner and combining (e.g., registering and stitching) the scan data to generate a 3D digital representation (e.g., a 3D model) of the intraoral structure. The method can include evaluating scanning accuracy of the scanner based on the results of the at least one intraoral scanning session, and outputting an indication of the scanning accuracy on a display. The scanning accuracy can be evaluated in various ways, such as by detecting the presence of stitching errors, detecting the presence of erroneous intersections between portions of the 3D digital representation, comparing 3D digital representations generated with different scan data to check for discrepancies, and/or by comparing the 3D digital representation to other data types (e.g., cone beam computed tomography (CBCT) data). Scanning accuracy can be evaluated based on the results of a single intraoral scanning session or based on the results from multiple intraoral scanning sessions. In some embodiments, the results of multiple intraoral scanning sessions are analyzed to identify changes in accuracy of a single scanner over time and/or to evaluate the differences in accuracy associated with different scanner types, scanning protocols, scanner manufacturing methods, scanning calibration methods, scanning software, etc.

The present technology can provide numerous advantages compared to conventional systems and methods for intraoral scanning. For instance, conventional scanner systems generally lack the capability to detect deteriorating scanning accuracy and/or to identify the source of accuracy issues. The systems and methods herein can use data generated during one or more scanning sessions to identify whether a particular scanner is exhibiting accuracy issues and whether corrective action (e.g., additional scanning and/or rescanning, recalibration, repair, replacement) should be taken. Accuracy monitoring can be performed at the location of the scanner (e.g., at a dental office), at a remote location (e.g., at a facility of a manufacturer of the scanner), or a combination thereof. Moreover, the techniques described herein can be used to assess differences in accuracy over time and/or between different scanner types, different scanners of the same type, different scanning protocols, different manufacturing methods, different calibration methods, different software versions, different users, etc., which may be beneficial for determining the cause of accuracy issues. In some instances, accuracy results are determined for a very large number of scanners and/or scans, which may make it possible to detect very slight and/or rare changes in accuracy that may be difficult or impossible to detect during normal quality control processes.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” “lower,” “left,” “right,” etc., can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Overview of Intraoral Scanning Technology

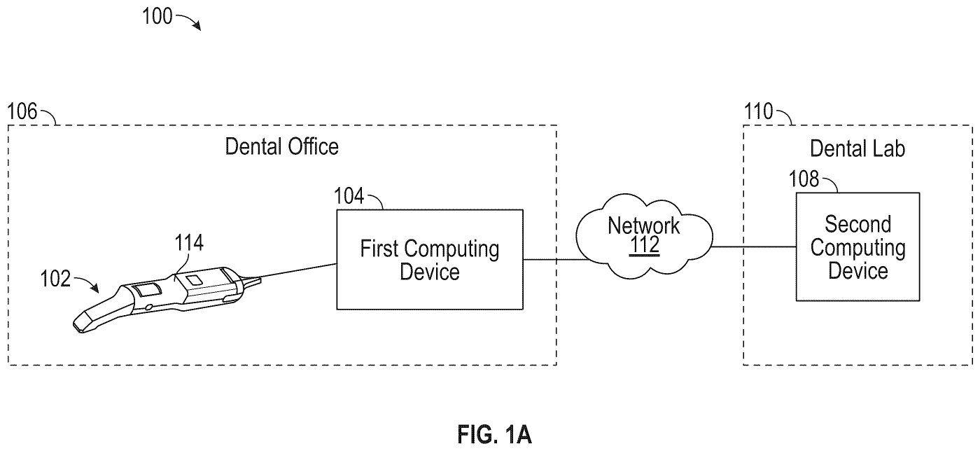

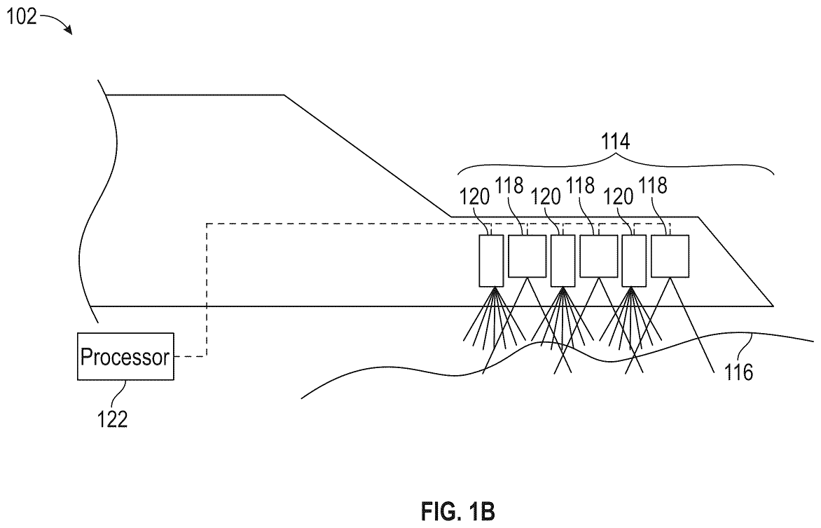

FIG. 1A schematically illustrates a system 100 for performing intraoral scanning and/or generating 3D digital representations of a patient's intraoral cavity, in accordance with embodiments of the present technology. The system 100 includes an intraoral scanner 102 (also referred to as a “scanner”) operably coupled to a first computing device 104. The scanner 102 and first computing device 104 can be at a first location, such as a dental office 106. Optionally, the first computing device 104 may be operably coupled to another second computing device 108 at a second location, such as a dental lab 110. The first computing device 104 and the second computing device 108 can be connected to one another via a network 112, such as a local area network (LAN), a public wide area network (WAN) (e.g., the Internet), a private WAN (e.g., an intranet), or a combination thereof.

The scanner 102 may be used to generate scan data of one or more intraoral structures of a patient, such as the teeth, gingiva, palate, tongue, checks, etc. The scan data can include 3D intraoral scans that provide a digital representation of the surface topography of the intraoral structures. For instance, the 3D intraoral scans can include one or more point clouds, height/depth maps, or any other suitable digital data format depicting the 3D geometry of the intraoral structures. Optionally, the scan data can include other types of digital data, such as color images and/or images obtained at various wavelengths (e.g., near-infrared (NIR) images, infrared images, ultraviolet images, fluorescent images). In some embodiments, the scanner 102 alternates between generation of 3D intraoral scans and one or more types of 2D intraoral images (e.g., color images, NIR images, infrared images, ultraviolet images, fluorescent images) during scanning.

In some embodiments, the scanner 102 includes a probe 114 (e.g., a handheld wand) that may be inserted at least partially into the intraoral cavity. The probe 114 can include or be coupled to one or more optical elements for outputting light toward intraoral structures and optically capturing features (e.g., surface topography, color) of the intraoral structures, such as one or more imaging devices (e.g., cameras), light sources (e.g., projectors, lasers), image sensors (e.g., CCD sensors, CMOS sensors), focusing optics (e.g., confocal optics), mirrors, prisms, lenses, beam splitters, polarizers, etc. The probe 114 can include a transparent or translucent window to allow light to pass out of the probe 114 toward the intraoral structures, and to allow light from the intraoral structures to be received by the probe 114.

FIG. 1B is a partially schematic illustration of an example scanner 102 that may be used in the system 100 of FIG. 1A, in accordance with embodiments of the present technology. The scanner 102 can be used to obtain scan data of an intraoral surface 116. In some embodiments, the scanner 102 includes a probe 114 at a distal end of the scanner 102. One or more cameras 118 are disposed within the probe 114 (e.g., rigidly fixed within the probe 114) and arranged within the probe 114 such that the cameras 118 receive rays of light from an intraoral cavity in a non-central manner (e.g., the relationship between points in 3D world-coordinate space and corresponding points on the camera sensors of the one or more cameras 118 is described by a set of camera rays for which there is no single point in space through which all of the camera rays pass).

In some embodiments, the scanner 102 is configured to perform intraoral scanning using structured light illumination. In such embodiments, one or more structured light projectors 120 can be disposed within the probe 114 and can project a pattern of structured light (e.g., a pattern of spots) onto the intraoral surface 116. Each camera 118 can be configured to capture a plurality of images that depict at least a portion of the projected pattern of structured light on the intraoral surface 116. In some embodiments, the structured light projectors 120 and cameras 118 are arranged in a closely packed and/or alternating fashion, such that a substantial part of each camera's field of view overlaps the field of view of neighboring cameras 118, and a substantial part of each projector's field of illumination overlaps the field of illumination of neighboring projectors 120. The positioning of the projectors 120 and the cameras 118 within the probe 114 can allow the scanner 102 to have an overall large field of view while maintaining a low profile probe geometry.

The scanner 102 can further include a processor 122 configured to generate a 3D model of the intraoral surface 116 based on images from one or more cameras 118. In some embodiments, the processor 122 solves a “correspondence problem,” where a correspondence between pattern elements in the structured light pattern and pattern elements seen by a camera 118 viewing the pattern is determined. The processor 122 may compensate for the image distortion specifically introduced by the non-central manner in which one or more cameras 118 receive rays of light from the intraoral surface 116 by altering the coordinates of one or more of the structured light pattern elements as seen by one or more cameras 118 in order to account for the non-central manner in which the one or more cameras 118 receive rays of light from the intraoral surface 116.

Referring again to FIG. 1A, other types of scanners 102 can be used in the system 100, alternatively or in addition to the embodiment of FIG. 1B. For instance, in some embodiments, the scanner 102 can be a confocal imaging apparatus including a light source that emits an array of light beams. The light source can be located at a proximal end of the probe 114, and the probe 114 can define a light transmission path from the proximal end of the probe 114 to the distal end of the probe. A set of confocal focusing optics may be positioned along the light transmission path between the proximal end and distal end of the probe 114. At the distal end, the probe 114 can include a mirror that directs the array of light beams towards an object outside of the scanner 102. The light beams reflected off the object can pass back into the probe 114 and be directed onto an image sensor. In some embodiments, the image sensor detects light intensity at each pixel, which may be used to compute height or depth.

Optionally, the scanner 102 may include other components, such as a movement sensor for measuring movement and/or pose of the scanner 102. For example, the movement sensor can be an internal measurement unit (IMU) (e.g., a micro-electromechanical system (MEMS) IMU), which may include one or more accelerometers, gyroscopes, magnetometers, pressure sensors, etc. As another example, the scanner 102 can include a temperature sensor and temperature control circuitry for measuring and controlling the temperature within the probe 114, e.g., to reduce fogging of optical elements and/or avoid patient discomfort. In a further example, the scanner 102 may be used in conjunction with a removable sleeve or other protective device that fits over the probe 114 to avoid contamination and/or for patient protection. The sleeve may be a single-use component or may be reusable.

Representative examples of intraoral scanners that may be used as the scanner 102 are described in U.S. Pat. Nos. 11,563,929 and 11,896,461, the disclosures of each of which are incorporated by reference herein in their entirety.

The scanner 102 can be coupled to the first computing device 104 via a wired or wireless connection. In some embodiments, the scanner 102 is wirelessly connected to the first computing device 104 via a direct wireless connection. In some embodiments, the scanner 102 is wirelessly connected to the first computing device 104 via a wireless network, such as a Wi-Fi network, Bluetooth network, a Zigbee network, or other wireless network. For example, the first computing device 104 may be physically connected to one or more wireless access points and/or wireless routers (e.g., Wi-Fi access points/routers), and the scanner 102 may include a wireless module (e.g., a Wi-Fi module) for joining the wireless network via the wireless access point and/or router.

The scan data obtained by the scanner 102 may be transmitted to the first computing device 104, and the first computing device 104 may store the scan data in a data store. The data store may include local data stores and/or remote data stores. The first computing device 104 can be a personal computer, workstation, laptop, tablet, smartphone, etc., that includes one or more processors, memory, secondary storage devices, input devices (e.g., a keyboard, mouse, tablet, touchscreen, microphone, camera), output devices (e.g., display, printer, touchscreen, speakers), and/or other suitable hardware components. The first computing device 104 can also include software components for monitoring and controlling the scanner 102, receiving and processing the scan data, and/or other functionality relevant to an intraoral scanning procedure.

In some embodiments, a user (e.g., a patient, a clinician, technician, or other practitioner) performs intraoral scanning of a patient in connection with a dental procedure. By way of non-limiting example, dental procedures may be broadly divided into prosthodontic (restorative) and orthodontic procedures, and then further subdivided into specific forms of these procedures. Additionally, dental procedures may include identification and treatment of periodontal disease, sleep apnea, and intraoral conditions. The term prosthodontic procedure may refer to any procedure involving the oral cavity and directed to the design, manufacture, or installation of a dental prosthesis at a dental site within the oral cavity, or a real or virtual model thereof, or directed to the design and preparation of the dental site to receive such a prosthesis. A prosthesis may include any restoration such as crowns, veneers, inlays, onlays, implants and bridges, for example, and any other artificial partial or complete denture. The term orthodontic procedure may refer to any procedure involving the intraoral cavity and directed to the design, manufacture, or installation of orthodontic elements at a dental site within the intraoral cavity, or a real or virtual model thereof, or directed to the design and preparation of the dental site to receive such orthodontic elements. These elements may be appliances including but not limited to brackets and wires, retainers, aligners, palatal expanders, attachment placement templates, mouth guards, oral sleep apnea devices, or other dental appliances.

In some embodiments, intraoral scanning is performed on a patient's intraoral cavity during a visitation of the dental office 106. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental and/or periodontal diagnostics, to generate a treatment plan, to determine progress of a treatment plan, and/or for other purposes.

During an intraoral scanning procedure, the user may apply the scanner 102 to one or more locations within the intraoral cavity of the patient. The scanning may be divided into one or more segments. As an example, the segments may include a lower dental arch of the patient (e.g., the complete lower dental arch or a portion thereof), an upper dental arch of the patient (e.g., the complete upper dental arch or a portion thereof), and/or patient bite (e.g., scanning performed with closure of the patient's mouth with the scan being directed towards an interface area of the patient's upper and lower teeth). Via such scanner application, the scanner 102 may provide scan data to the first computing device 104. The scan data may be provided in the form of intraoral scan data sets, each of which may include 3D intraoral scans (e.g., point clouds, height/depth maps) and/or 2D intraoral images (e.g., color images, NIR images, infrared images, ultraviolet images, fluorescent images).

The first computing device 104 can include one or more software components configured to process the scan data into a 3D digital representation of the patient's intraoral structures. For example, the first computing device 104 can implement an intraoral scan application that registers and stitches together two or more intraoral scans from the scan data to generate a growing 3D surface. In some embodiments, performing registration includes capturing 3D data of various points of a surface in multiple scans, and registering the scans by computing transformations between the scans (e.g., based on overlapping points depicted in the scans). One or more 3D surfaces may be generated based on the registered and stitched together intraoral scans during the intraoral scanning. The one or more 3D surfaces may be output to a graphical user interface (GUI) on a display of the first computing device 104 so that the user can view the scan progress thus far. As each new intraoral scan is captured and registered to previous intraoral scans and/or to the generated 3D surface(s), the 3D surface(s) may be updated, and the updated 3D surface(s) may be output to the display. The user interface showing the 3D surface(s) may be periodically or continuously updated to show scanning progress in real time or near-real time.

When a scan session or a portion of a scan session associated with a particular scanning segment (e.g., upper dental arch, lower dental arch, bite) is complete (e.g., all scans for the site of interest have been captured), the intraoral scan application may generate a 3D digital representation of the scanned segment (e.g., a virtual 3D model). The 3D digital representation may be a set of 3D points and their connections with each other (e.g., a mesh). To generate the 3D digital representation, the intraoral scan application may register and stitch together the intraoral scans generated from the intraoral scan session that are associated with a particular scanning segment. The registration performed at this stage may be more accurate than the registration performed during the capturing of the intraoral scans, and may take more time to complete than the registration performed during the capturing of the intraoral scans. In some embodiments, performing scan registration includes capturing 3D data of various points of a surface in multiple scans, and registering the scans by computing transformations between the scans. The 3D data may be projected into a 3D space of the 3D digital representation to form a portion of the 3D digital representation. The intraoral scans may be integrated into a common reference frame by applying appropriate transformations to points of each registered scan and projecting each scan into the 3D space.

In some embodiments, registration is performed for adjacent or overlapping intraoral scans (e.g., each successive frame of an intraoral video). Registration algorithms may be carried out to register two adjacent or overlapping intraoral scans and/or to register an intraoral scan with a 3D digital representation, which can involve determination of the transformations which align one scan with the other scan and/or with the 3D digital representation. Registration may involve identifying multiple points in each scan (e.g., point clouds) of a scan pair (or of a scan and the 3D digital representation), surface fitting to the points, and using local searches around points to match points of the two scans (or of the scan and the 3D digital representation). For example, the intraoral scan application may match points of one scan with the closest points interpolated on the surface of another scan, and iteratively minimize the distance between matched points. Other registration techniques known to those of skill in the art may also be used. Examples of registration techniques include, for example, iterative closest point (ICP) algorithms.

The intraoral scan application may repeat registration for all intraoral scans of a sequence of intraoral scans to obtain transformations for each intraoral scan, to register each intraoral scan with previous intraoral scan(s) and/or with a common reference frame (e.g., with the 3D digital representation). Intraoral scan application may integrate intraoral scans into a single 3D digital representation by applying the appropriate determined transformations to each of the intraoral scans. Each transformation may include rotations about one to three axes and/or translations along one to three axes.

The intraoral scan application may generate one or more 3D digital representations from intraoral scans, and may display the 3D digital representation(s) to the user via a GUI on the display. The 3D digital representation(s) can then be checked visually by the user. The user can virtually manipulate the 3D digital representation(s) via the user interface with respect to up to six degrees of freedom (e.g., translated and/or rotated with respect to one or more of three mutually orthogonal axes) using suitable user controls (e.g., hardware and/or software controls) to enable viewing of the 3D digital representation(s) from any desired direction.

Optionally, the scan data generated by the scanner 102 and/or the 3D digital representation(s) generated by the intraoral scan application may be transmitted from the first computing device 104 to the second computing device 108 via the network 112. The second computing device 108 can be coupled to a data store for storing the scan data and/or the 3D digital representations, which may include local data stores and/or remote data stores. The second computing device 108 can be a personal computer, workstation, laptop, tablet, smartphone, etc., that includes one or more processors, memory, secondary storage devices, input devices (e.g., a keyboard, mouse, tablet, touchscreen, microphone, camera), output devices (e.g., display, printer, touchscreen, speakers), and/or other suitable hardware components.

In some embodiments, the second computing device 108 includes one or more software components configured to perform dental and/or periodontal diagnostics, generate a treatment plan, to determine progress of a treatment plan, and/or for other purposes relevant to a dental procedure, based on the scan data and/or the 3D digital representation(s). For example, a 3D digital representation of a patient's intraoral cavity may be used to design a dental prosthesis for a prosthodontic procedure, such as one or more crowns, veneers, inlays, onlays, implants, bridges, etc. As another example, a 3D digital representation of a patient's intraoral cavity may be used to design a dental appliance for an orthodontic procedure, such as one more aligners, retainers, palatal expanders, etc. In a further example, a 3D digital representation of a patient's intraoral cavity may be used to diagnose a patient with periodontal disease, sleep apnea, and/or other intraoral conditions.

In some embodiments, one or more 3D digital representations can be used for designing dental appliances, such as aligners and/or a series of aligners with tooth-receiving cavities configured to move a person's teeth from an initial arrangement toward a target arrangement in accordance with a treatment plan. Aligners can include mandibular repositioning elements, such as those described in U.S. Pat. No. 10,912,629, entitled “Dental Appliances with Repositioning Jaw Elements,” filed Nov. 30, 2015; U.S. Pat. No. 10,537,406, entitled “Dental Appliances with Repositioning Jaw Elements,” filed Sep. 19, 2014; and U.S. Pat. No. 9,844,424, entitled “Dental Appliances with Repositioning Jaw Elements,” filed Feb. 21, 2014; all of which are incorporated by reference herein in their entirety.

One or more 3D digital representations can also be used to design attachment placement devices, e.g., appliances used to position prefabricated attachments on a person's teeth in accordance with one or more aspects of a treatment plan. Examples of attachment placement devices (also known as “attachment placement templates” or “attachment fabrication templates”) can be found at least in: U.S. application Ser. No. 17/249,218, entitled “Flexible 3D Printed Orthodontic Device,” filed Feb. 24, 2021; U.S. application Ser. No. 16/366,686, entitled “Dental Attachment Placement Structure,” filed Mar. 27, 2019; U.S. application Ser. No. 15/674,662, entitled “Devices and Systems for Creation of Attachments,” filed Aug. 11, 2017; U.S. Pat. No. 11,103,330, entitled “Dental Attachment Placement Structure,” filed Jun. 14, 2017; U.S. application Ser. No. 14/963,527, entitled “Dental Attachment Placement Structure,” filed Dec. 9, 2015; U.S. application Ser. No. 14/939,246, entitled “Dental Attachment Placement Structure,” filed Nov. 12, 2015; U.S. application Ser. No. 14/939,252, entitled “Dental Attachment Formation Structures,” filed Nov. 12, 2015; and U.S. Pat. No. 9,700,385, entitled “Attachment Structure,” filed Aug. 22, 2014; all of which are incorporated by reference herein in their entirety.

One or more 3D digital representations can be used to design incremental palatal expanders and/or a series of incremental palatal expanders used to expand a person's palate from an initial position toward a target position in accordance with one or more aspects of a treatment plan. Examples of incremental palatal expanders can be found at least in: U.S. application Ser. No. 16/380,801, entitled “Releasable Palatal Expanders,” filed Apr. 10, 2019; U.S. application Ser. No. 16/022,552, entitled “Devices, Systems, and Methods for Dental Arch Expansion,” filed Jun. 28, 2018; U.S. Pat. No. 11,045,283, entitled “Palatal Expander with Skeletal Anchorage Devices,” filed Jun. 8, 2018; U.S. application Ser. No. 15/831,159, entitled “Palatal Expanders and Methods of Expanding a Palate,” filed Dec. 4, 2017; U.S. Pat. No. 10,993,783, entitled “Methods and Apparatuses for Customizing a Rapid Palatal Expander,” filed Dec. 4, 2017; and U.S. Pat. No. 7,192,273, entitled “System and Method for Palatal Expansion,” filed Aug. 7, 2003; all of which are incorporated by reference herein in their entirety.

The system 100 can be configured in many different ways. For example, any of the components of the system 100 shown as distinct elements in FIG. 1A can be combined into a single device, and/or any of the components of the system 100 shown as a single element in FIG. 1A can be divided into a plurality of discrete devices. Moreover, the locations of the components of the system 100 can be varied as desired, e.g., any of the components shown in FIG. 1A can be located at the dental office 106, the dental lab 110, or at one or more other locations, such as a server farm that provides a cloud computing service, a facility of a manufacturer of the scanner 102, a facility of a manufacturer of dental appliances and/or dental prosthetics, etc. Additionally, any of the operations that are described as being performed by a particular component of the system 100 can alternatively or additionally be performed by any other component of the system 100, e.g., the operations of the first computing device 104 may alternatively or additionally be performed by the second computing device 108 and/or by another computing device (e.g., a remote server), and vice-versa.

The system 100 may include additional components not illustrated in FIG. 1A. For instance, although FIG. 1A depicts a single scanner 102, the system 100 can optionally include multiple scanners 102, which may be at the same location (e.g., the same dental office 106) or at different locations (e.g., different dental offices 106). Similarly, although FIG. 1A depicts a single dental office 106 and a single dental lab 110, the system 100 may include multiple dental offices 106, multiple dental labs 110, and/or other facilities including respective computing devices that are communicably coupled to each other via one or more networks 112 in any suitable arrangement.

### II. Monitoring Scanning Accuracy

In some embodiments, the present technology provides methods and systems for monitoring accuracy of a scanner. Poor scanning accuracy may arise due to a variety of reasons, such as hardware malfunction (e.g., parts may become misaligned, broken, degraded, etc.; scanner components such as the sleeve and/or window may become fouled or damaged, e.g., due to reuse of single-use components, scratching, exposure to biological fluids), software issues (e.g., errors introduced by software updates or new software versions), issues related to the computing device for the scanner (e.g., computer performance may be compromised due to lack of disk space, viruses, CPU slowdown due to heating, etc.; there may be issues specific to certain computer types (e.g., laptop versus personal computer), computer models, computer processors, computer processor types, etc.), calibration drift over time, noise, and/or user error (e.g., if scanning is not performed according to recommended protocols). In embodiments where scan data is used for diagnostic and/or therapeutic purposes (e.g., diagnosing a patient with an intraoral condition, designing dental appliances to treat the patient's teeth, monitoring progress of a dental treatment), deteriorating scanning accuracy can compromise the accuracy and efficacy of the diagnosis and/or therapy (e.g., the patient may be misdiagnosed, the dental appliances may not fit properly on the patient's teeth, progress tracking may be inaccurate).

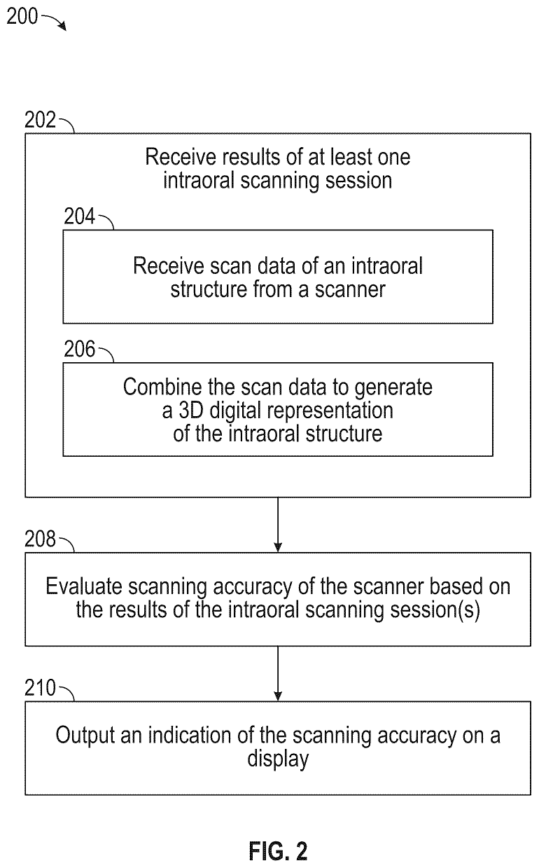

FIG. 2 is a flow diagram illustrating a method 200 for monitoring scanning accuracy, in accordance with embodiments of the present technology. The method 200 can be implemented with any of the scanner systems described herein, such as the system 100 of FIGS. 1A and 1B. In some embodiments, some or all of the processes of the method 200 are implemented as computer-readable instructions (e.g., program code) that are configured to be executed by one or more processors of a computing device (e.g., a client device, a server device, or suitable combinations thereof). For example, any of the processes of the method 200 can be performed by the first computing device 104 and/or the second computing device 108 of the system 100 of FIGS. 1A and 1B.

The method 200 can begin at block 202 with receiving results of at least one intraoral scanning session. An intraoral scanning session can include receiving scan data of an intraoral structure (sub-block 204), then combining the scan data to generate a 3D digital representation of the intraoral structure (sub-block 206). For example, the intraoral structure can include the patient's teeth, gingiva, palate, tongue, cheeks, etc. The intraoral structure can include an upper dental arch, a lower dental arch, or both dental arches. In some embodiments, multiple intraoral structures are scanned sequentially. For instance, scanning of a dental arch may proceed by first scanning the occlusal surfaces (occlusal scan), then scanning the lingual surfaces (lingual scan), then scanning the buccal surfaces (buccal scan). Scanning of both dental arches may proceed by first scanning the lower dental arch, then scanning the upper arch, then scanning the lower and upper dental arches in occlusion with each other (bite scan). Other variations of the scanning procedure may also be used, depending on clinician preference, patient anatomy, the regions of interest, etc.

The intraoral scanning session may be performed using any suitable scanner, such as any of the embodiments described herein (e.g., in Section I above). The scan data produced by the scanner can include 3D scans (e.g., point clouds, height/depth maps), 2D images (e.g., color images, NIR images, infrared images, ultraviolet images, fluorescent images), and/or any other digital data providing a digital representation of the characteristics of the scanned intraoral structures. The type and format of scan data produced may vary according to the scanner type, clinician preference, the purpose of the scan data (e.g., diagnosis, treatment planning, progress tracking), the type of intraoral structure to be scanned, etc. The scan data can be registered and stitched together to produce a 3D digital representation (e.g., 3D surface model) of the intraoral structure. The results of the intraoral scanning session can include the scan data, the 3D digital representation generated from the scan data, and/or any other information obtained in connection with the intraoral scanning session (e.g., user feedback on perceived scanning accuracy, difficulty of the scanning conditions).

At block 208, the method 200 can continue with evaluating scanning accuracy of the scanner, based on the results of the intraoral scanning session(s). For example, scanning accuracy can be evaluated based on the 3D digital representation (e.g., the entire 3D digital representation or a portion thereof), the scan data used to generate the 3D digital representation (e.g., one or more individual scans), intermediate data produced during the process of the generating the 3D digital representation (e.g., an incomplete portion of the 3D digital representation during registration and stitching), or suitable combinations thereof. For example, in some instances, the 3D digital representation only may not be sufficient for evaluating scanning accuracy, and may be supplemented with additional data to improve the accuracy of the evaluation.

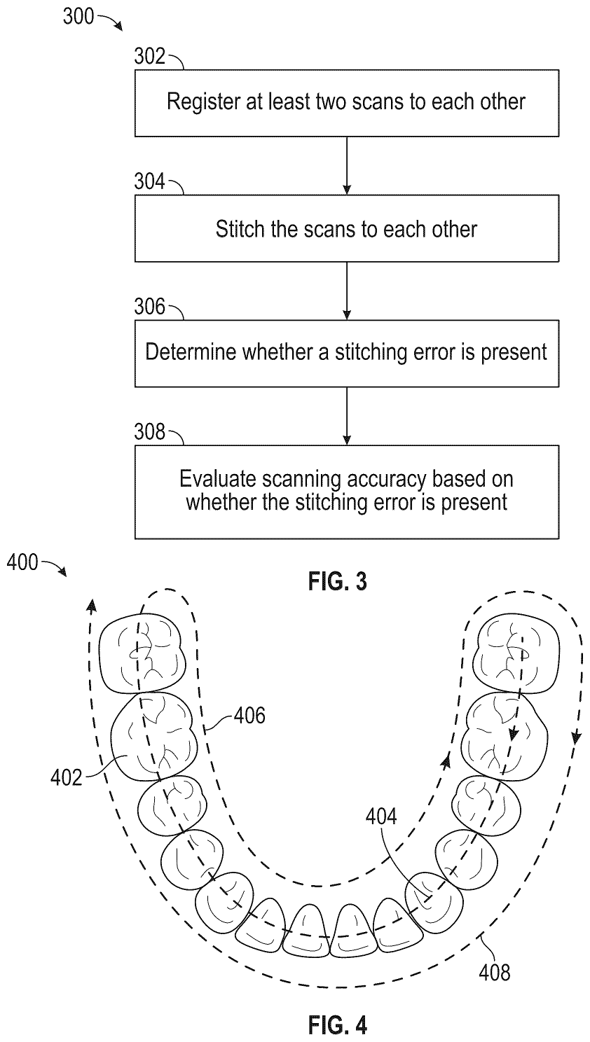

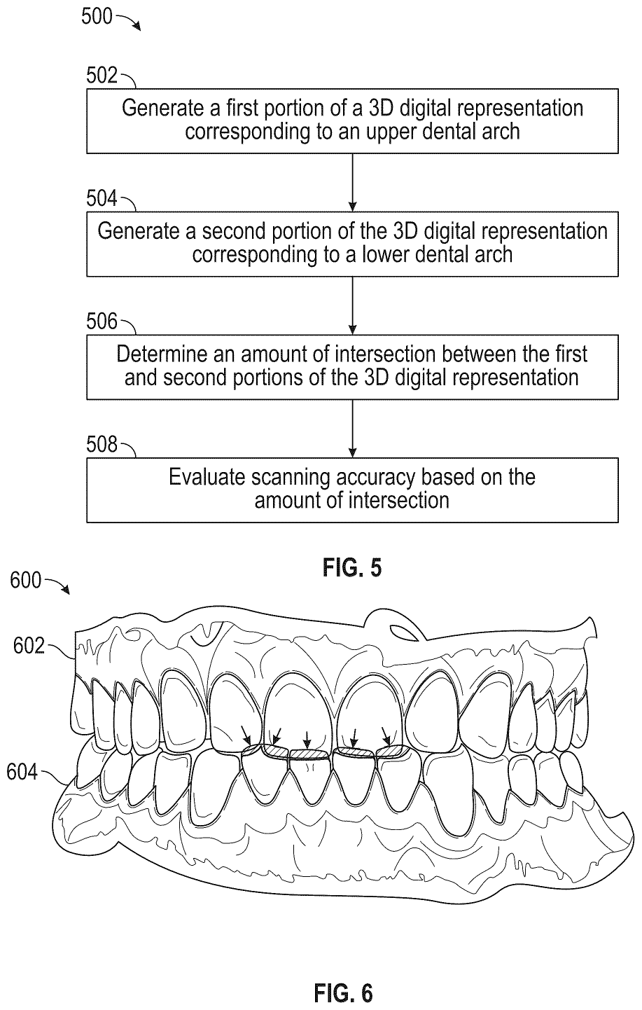

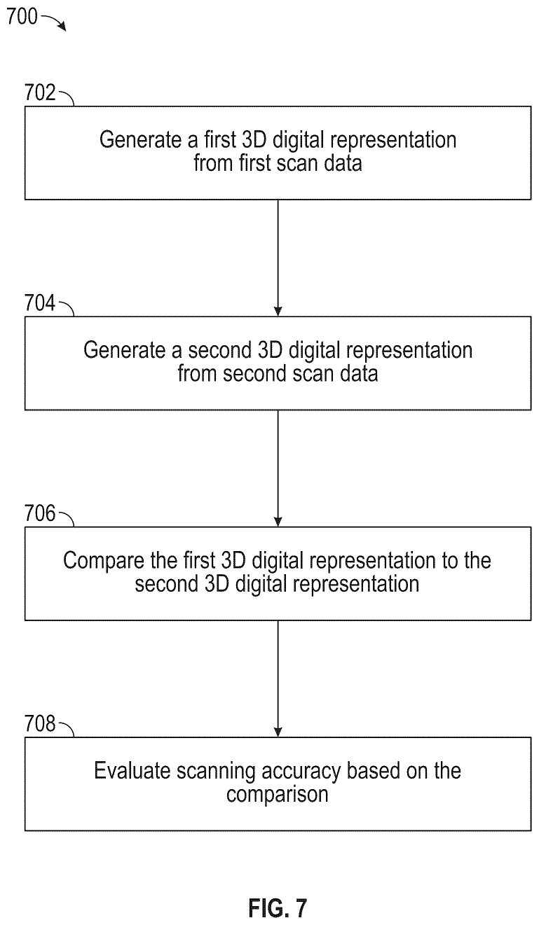

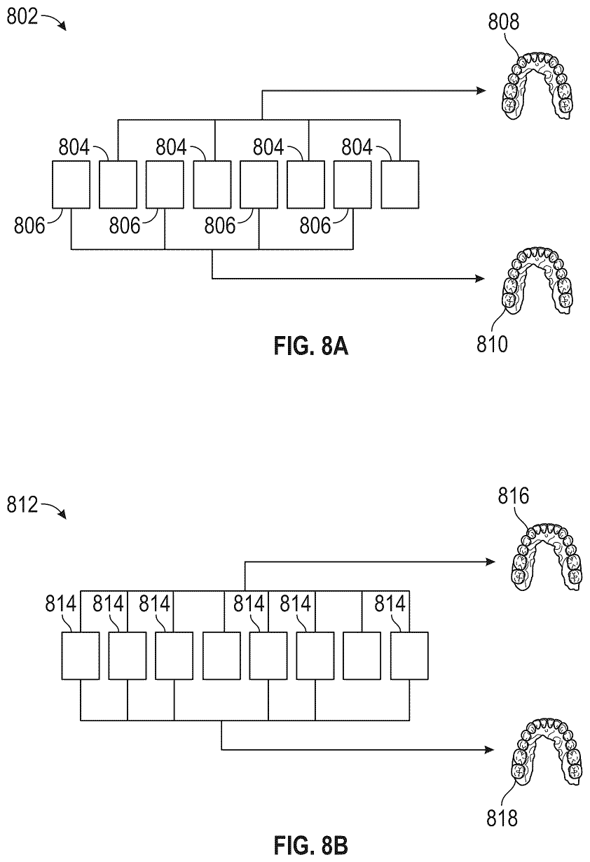

The scanning accuracy can be evaluated using one or more techniques. For example, in embodiments where the intraoral scanning session(s) involve registering and stitching multiple scans together to generate a 3D digital representation, the scanning accuracy can be evaluated based on an amount of error in the registration and/or stitching process, e.g., as discussed further below in connection with FIGS. 3 and 4. As another example, the scanning accuracy can be evaluated based on whether the 3D digital representation generated from the intraoral scanning session includes features that are anatomically and/or physically impossible or implausible, such as intersections between the upper and lower dental arches, e.g., as discussed further below in connection with FIGS. 5 and 6. In a further example, the scanning accuracy can be evaluated by comparing 3D digital representations generated from different scan data, e.g., as described further below in connection with FIGS. 7-8B.

In yet another example, the scanning accuracy can be evaluated based on additional data besides the results of the intraoral scanning session(s). The additional data can include other types of data of the intraoral structure, such as photographs, video, magnetic resonance imaging (MRI) data, and/or radiographic data (e.g., standard x-ray data such as bitewing x-ray data, panoramic x-ray data, cephalometric x-ray data, computed tomography (CT) data, cone-beam computed tomography (CBCT) data, fluoroscopy data). For instance, CBCT data of patient's intraoral cavity may be compared to the scanning results, and significant discrepancies between the CBCT data and scanning results may be indicative of accuracy issues. Alternatively or in combination, the additional data can include results from scanning a reference object having known dimensions and/or distances, such as a 4-sphere jaw model, a restorative object in the patient's intraoral cavity, etc. Significant deviations between the results from scanning the reference object and the known geometry of the reference object may be indicative of accuracy issues. In some embodiments, the additional data can include user feedback, e.g., the user's assessment of whether the scanning results were accurate or not. For instance, a user of the scanner may provide input stating whether they believe there are accuracy issues with the scanner. The user feedback can also include feedback regarding the difficulty of the scanning conditions, such as whether the intraoral structure was easy or difficult to scan, the types of intraoral structure scanned, the type of scanning performed, the duration of the intraoral scanning session, etc.

The scanning accuracy may be evaluated based on results from a single intraoral scanning session or from a plurality of intraoral scanning sessions. In embodiments where the results are from multiple intraoral scanning sessions, some or all of the intraoral scanning sessions may differ from each other with respect to any of the following: scanner type, scanning protocol, scanner manufacturing method, scanning calibration method, scanning software (e.g., different software versions), and/or user (e.g., different clinicians). For instance, scanning accuracy of the same scanner may be evaluated at multiple different time points to detect changes in accuracy of the scanner over the lifetime of the scanner, e.g., due to deteriorating hardware, changes in software versions, issues related to the computing device for the scanner, calibration drift, different users and/or scanning protocols, etc. As another example, differences in scanning accuracy between different scanner types, between different scanners of the same type, between different scanning protocols, different manufacturing and/or calibration methods, different manufacturing batches, different software versions, different users, different computer processors, different computer processor types, different sleeves, different sleeve types, etc., can be assessed to generate accuracy statistics across different conditions and/or identify potential sources of accuracy issues. In some embodiments, scanning accuracy is evaluated across a large volume of scan data and/or a large number of scanning sessions to detect small and/or rare changes in accuracy that may not be detectable through normal quality control procedures.

Scanning accuracy may be evaluated quantitatively, qualitatively, or both. For instance, scanning accuracy may be quantified based on an error value, e.g., an error value corresponding to a discrepancy between the actual object distance and the distance in the scanning results; an error value corresponding to a discrepancy between two sets of scanning results that are supposed to be the same or similar; whether the error value exceeds a threshold indicative of inadequate scanning accuracy; the frequency at which the error value exceeds the threshold value; changes in the error value over time and/or across different conditions; etc. Qualitative assessments of scanning accuracy can include ratings (e.g., “high accuracy,” “moderate accuracy,” “poor accuracy”), descriptive statements, classifications, etc.

Optionally, the process of block 208 can further include determining a confidence level for the scanning accuracy. The confidence level may depend on the amount of scanning results that are available, the length of time over which the scanning results are obtained, the number of intraoral scanning sessions used to generate the scanning results, the type and/or magnitude of the detected errors, etc. For instance, scanning accuracy determined from a larger number of scanning results may have a higher confidence level than scanning accuracy determined from a smaller number of scanning results. As another example, large amounts of scan data may provide more confident detection of smaller and/or more rare errors, whereas small amounts of scan data may provide confident detection for large and/or frequent errors only.

In some embodiments, the determined scanning accuracy may be normalized or otherwise adjusted based on the difficulty level of the intraoral scanning session. For instance, certain types of intraoral objects may be more difficult to scan accurately than others, e.g., an edentulous jaw may have fewer features for the registration and stitching algorithms, a smaller jaw may have less accumulated errors than a larger jaw, etc. Additionally, the scanning protocol can also affect accuracy, e.g., a slower scan with larger amounts of scan data of an object of interest may be easier to register and stitch compared to a faster scan that may skip or miss certain areas, the distance between the object and scanner may also affect the accuracy of the scan data, etc. Moreover, scanning accuracy may vary depending on the material and/or conditions of the object of interest, e.g., intraoral structures that are covered in saliva and/or have highly reflective surfaces may be more difficult to scan accurately compared to intraoral structures that are dry and/or have less reflective surfaces.

Accordingly, the process of block 208 can involve adjusting the scanning accuracy based on the difficulty level. In some embodiments, difficulty level information is obtained based on user input, scan data, output of the registration and/or stitching algorithms (e.g., a confidence level output by the algorithm), and/or other suitable techniques. The scanning accuracy can be adjusted in various ways. For example, in embodiments where the scanning accuracy is or includes an error value, the error value can be normalized, e.g., by outputting the error value as a percentage of the overall object size and/or overall distance scanned, rather than as an absolute distance measurement. In some embodiments, an expected error level can be determined for various scanning conditions and/or difficulty levels, and the error value can be normalized to the expected error level. Optionally, rather than adjusting the scanning accuracy, the method 200 can provide the difficulty level information together with the scanning accuracy to provide greater context.

At block 210, the method 200 can include outputting an indication of the scanning accuracy on a display. The indication can be a textual and/or numerical representation of the scanning accuracy, such as one or more error values, error rates, confidence levels, statistics, ratings, descriptive statements, classifications, etc. Alternatively or in combination, the indication can be a graphical representation of the scanning accuracy, e.g., a graph showing statistics and/or trends in accuracy over time, across different scanner types, etc.; a visualization identifying variations in accuracy at different locations in the 3D digital representation, such as a heat map of error values; etc. In some embodiments, the indication provides a notification to a user (e.g., the user operating the scanner), such as an alert that poor scanning accuracy has been detected (e.g., if the error value exceeds a threshold). Optionally, the indication can provide a recommendation for one or more actions to be taken. For example, if poor scanning accuracy is detected, the recommendation can include any of the following: repeating an intraoral scanning session with the scanner (e.g., if the scanning accuracy for that particular session is adequate), recalibrating the scanner, repairing the scanner, or replacing the scanner.

In some embodiments, if poor scanning accuracy is detected, the method 200 can further include determining a cause of the poor scanning accuracy, and outputting an indication of the determined cause on the display. As described herein, poor scanning accuracy may be attributable to various factors, such as hardware malfunction, software issues, computer issues, calibration drift, noise, and/or user error. The cause(s) of scanning accuracy may be determined by comparing scanning accuracy across different intraoral scanning sessions. For example, a gradual decrease in scanning accuracy over time across multiple sessions performed by the same scanner may indicate that the scanner is experiencing calibration drift. As another example, a sudden decrease in scanning accuracy across multiple sessions performed by the same scanner may indicate that the scanner has experienced a hardware malfunction (e.g., one or more scanner components have become damaged, degraded, fouled, misaligned, etc.). In a further example, a sudden decrease in scanning accuracy after a software update and/or a change in software version may indicate a software issue. In yet another example, accuracy issues attributable to a particular scanner, scanner type, manufacturing method, manufacturing facility, calibration method, scanning protocol, user, computer, scanner sleeve, etc., may be detected if the accuracy of scan data obtained under those conditions is consistently worse compared to scan data obtained under other conditions (e.g., a particular scanner type consistently produces poorer accuracy than other scanner types). By outputting an indication of the potential cause(s) of scanning accuracy, the method 200 allows a user (e.g., a user of the scanner, a manufacturer of the scanner) to take appropriate corrective action.

Moreover, in embodiments where the method 200 monitors scanning accuracy over a very large number of scans and/or intraoral scanning sessions, very slight and/or rare changes in accuracy can be detected. For instance, it may be difficult to reliably detect accuracy issues based on errors in a single scan or a small number of scans, particularly if the errors fall within normal variances. However, if errors are consistently present in a large volume of scans (e.g., tens or hundreds of thousands of scans) generated from multiple scanners, this can provide a reliable indication that accuracy issues are present, even if the magnitudes of the individual errors are small. Similarly, changes in other statistics over time such as variance, the number of high error scans, etc., can be more reliably detected based on larger volumes of scan data.

In some embodiments, the method 200 is performed during or immediately after an intraoral scanning session by a local client device, such as computing device at the location of the intraoral scanning session (e.g., “in the field” accuracy monitoring). For example, the first computing device 104 of the system 100 of FIGS. 1A and 1B may perform some or all of the processes of the method 200 in order to evaluate the accuracy of the scanner 102 during or after one or more intraoral scanning sessions with the scanner 102. If the scanning accuracy is inadequate, the first computing device 104 can notify a user of the scanner 102 (e.g., by outputting an alert or other notification on a display of the first computing device 104) and/or can instruct the user to take corrective action (e.g., repeating the scanning session, calibrate the scanner 102, repair or replace the scanner 102). The first computing device 104 can also notify another user that accuracy issues have been detected, such as a user associated with a manufacturer of the scanner 102, by transmitting an indication of the scanning accuracy to another computing device (e.g., the second computing device 108 or a remote server). Alternatively or in addition, the method 200 may be performed by a remote server or other computing device that is remote from the location of the intraoral scanning session. For example, the second computing device 108 of the system 100 of FIGS. 1A and 1B may perform some or all of the processes of the method 200 to evaluate whether the scan data received from the first computing device 104 is accurate. Scan data can also be transmitted to a computing device of a manufacturer of the scanner 102 so the manufacturer can monitor the accuracy of its products. In some embodiments, the manufacturer can track scanning accuracy across multiple scanners over time to identify issues attributable to particular scanner types, manufacturing methods, suppliers, software versions, users, computers, scanner sleeves, etc., in order to determine the appropriate response (e.g., recalling certain scanner types, pushing corrective software updates, changing manufacturing techniques, updating user training).

Optionally, the method 200 may be performed in response to a triggering event. The triggering event can be an indication that a particular scanner is believed to have poor scanning accuracy (e.g., based on feedback from a user of the scanner). The triggering event can be a request from a manufacturer of the scanner to monitor scanning accuracy of one or more scanners, e.g., in response to user reports of poor scanning accuracy, as part of routine quality controls implemented by the manufacturer, etc. In such embodiments, once the triggering event occurs, the method 200 can be performed to evaluate the scanning accuracy of one or more scanners in order to confirm whether accuracy issues are indeed present, determine the potential causes of accuracy issues, and/or provide recommendations for potential corrective actions.

FIGS. 3-8B illustrate various techniques for evaluating the accuracy of a scanner. The embodiments described in connection with FIGS. 3-8B may be implemented by the methods and systems for monitoring scanning accuracy described herein, such as the embodiments of FIGS. 1A-2. Moreover, any of the embodiments of FIGS. 3-8B may be used independently or in combination with each other.

FIG. 3 is a flow diagram illustrating a method 300 for evaluating scanning accuracy based on stitching errors, in accordance with embodiments of the present technology. The method 300 can be implemented with any of the scanner systems described herein, such as the system 100 of FIGS. 1A and 1B. In some embodiments, some or all of the processes of the method 300 are implemented as computer-readable instructions (e.g., program code) that are configured to be executed by one or more processors of a computing device (e.g., a client device, a server device, or suitable combinations thereof). For example, any of the processes of the method 300 can be performed by the first computing device 104 and/or the second computing device 108 of the system 100 of FIGS. 1A and 1B. The method 300 may be combined with any of the other methods described herein, such as the method 200 of FIG. 2.

The method 300 can begin at block 302 with registering at least two scans to each other. The scans can be point clouds, height/depth maps, or other data depicting the 3D topography of an intraoral structure, and may be part of scan data obtained using a scanner as described elsewhere herein. The registration can be performed using a registration algorithm that determines one or more transformations to align one scan with a consecutive scan (e.g., the scan taken at the next point in time), such as by identifying multiple points in each scan, surface fitting to the points, and using local searches around points to match points of the two scans. In some embodiments, the registration algorithm identifies portions of each scan that overlap with each other, and aligns the scans with each other so that the overlapping portions match.

At block 304, the method 300 can include stitching the scans to each other, based on the registration. The stitching can be performed by a stitching algorithm that aligns the scans to each other according to the transformations computed by the registration algorithm, and then digitally combines the scans to generate at least a portion of 3D digital representation of the intraoral structure (e.g., a growing 3D surface).

At block 306, the method 300 can continue with determining whether a stitching error is present. Stitching errors may arise, for example, if the registration and stitching processes fail to properly connect portions of the intraoral structure that are supposed to overlap or be connected to each other. The presence of stitching errors may be correlated to scanning accuracy, e.g., large stitching errors (e.g., greater than 50 μm, 100 μm, 200 μm, or 500 μm) may be attributable to inaccuracies in the scan data and thus may indicate that the accuracy of the scanner has been compromised.

In some embodiments, stitching errors are detected based on whether there are discrepancies in portions of the 3D digital representation that are supposed to represent the same locations of the intraoral structure, e.g., whether there are discontinuities (e.g., gaps, mismatches, inconsistencies) between portions of the 3D digital representation that are supposed to overlap or be connected with each other. Such stitching errors can be detected based on scans that are obtained while the scanner is moved over the same location on the intraoral structure along different directions and/or at different times in the scanning session. If the scan data is accurate, scans of the same location should match each other and should be able to be registered and stitched into a continuous 3D digital representation. A stitching error may be present if the scans do not match and/or cannot be registered and stitched to each other to generate a continuous 3D digital representation of the location of the intraoral structure due to accumulated errors in the scan data.

For example, a 3D digital representation can include a first portion generated from first scan data obtained while the scanner is moved over a location on the intraoral structure along a first direction (e.g., during a first segment of a scanning trajectory), and a second portion generated from second scan data obtained while the scanner is moved over the location on the intraoral structure along a second direction (e.g., during a second segment of the scanning trajectory). Stated differently, the scanning trajectory can be a “loop” trajectory in which multiple scanning passes of the same location of the intraoral structure are performed. In the absence of stitching errors, the first and second portions of the 3D digital representation should be consistent with each other (“loop closure”). If there is a discrepancy (e.g., a discontinuity) between the first and second portions of the 3D digital representation generated from the first and second scan data, respectively, this can be indicative of the presence of a stitching error. In some embodiments, the stitching error is determined based on stitching and registration of scans performed without using “loop closure” software algorithms, multiple iterative closest point (ICP) algorithms, or other algorithms that automatically detect and correct discrepancies in different scans of the same location.

FIG. 4 illustrates an example of a scanning trajectory 400 for detecting stitching errors, in accordance with embodiments of the present technology. The trajectory 400 can include moving a scanner over different locations of a dental arch 402 to generate scan data thereof. In the illustrated embodiment, the trajectory 400 includes a first segment 404 in which the scanner moves over the occlusal surfaces of the dental arch 402 (occlusal scan), a second segment 406 in which the scanner moves over the buccal surfaces of the dental arch 402 (buccal scan), and a third segment 408 in which the scanner moves over the lingual surfaces of the dental arch 402 (lingual scan). Accordingly, multiple scanning passes are performed for each tooth in the dental arch 402. If stitching errors are present, there may be discrepancies between scan data generated from different scanning passes over the same location, e.g., scans of the same location generated during the first segment 404, second segment 406, and/or third segment 408 of the trajectory 400 may fail to be properly registered and stitched to each other.

Referring again to FIG. 3, at block 308, the method 300 can include evaluating scanning accuracy based on whether the stitching error is present. As discussed herein, the presence of large stitching errors may be attributable to poor scanning accuracy. In some embodiments, the scanning accuracy is measured based on the stitching error, e.g., the magnitude of the stitching error can be used as the error value for the scanner. Optionally, the stitching error can be normalized to compensate for variations in scanning difficulty, e.g., the stitching error can be normalized as a percentage of the total distance traveled by the scanner. The scanner can be determined to have poor scanning accuracy if the stitching error exceeds a threshold value.

FIG. 5 is a flow diagram illustrating a method 500 for evaluating scanning accuracy based on bite penetration, in accordance with embodiments of the present technology. The method 500 can be implemented with any of the scanner systems described herein, such as the system 100 of FIGS. 1A and 1B. In some embodiments, some or all of the processes of the method 500 are implemented as computer-readable instructions (e.g., program code) that are configured to be executed by one or more processors of a computing device (e.g., a client device, a server device, or suitable combinations thereof). For example, any of the processes of the method 500 can be performed by the first computing device 104 and/or the second computing device 108 of the system 100 of FIGS. 1A and 1B. The method 500 may be combined with any of the other methods described herein, such as the method 200 of FIG. 2 and/or the method 300 of FIG. 3.

At block 502, the method 500 includes generating a first portion of a 3D digital representation corresponding to an upper dental arch, and at block 504, the method 500 includes generating a second portion of the 3D digital representation corresponding to a lower dental arch. The 3D digital representation can depict both jaws of the patient, with the first and second portions of the 3D digital representation showing the upper dental arch and lower dental arch, respectively, positioned in occlusion with each other. In some embodiments, the 3D digital representation is generated from (1) single jaw scans of the upper and lower dental arches and (2) a bite scan obtained with the patient's jaws closed.

At block 506, the method 500 can include determining an amount of intersection between the first and second portions of the 3D digital representation. In the absence of scanning errors, the first and second portions should reflect the actual occlusion between the upper and lower dental arches. However, if scanning errors are present, the digital representations of the upper and lower dental arches may intersect each other (“bite penetration”). The amount of intersection can be quantified based on the intersection distance between the upper and lower dental arches (e.g., an average and/or maximum intersection distance). In some embodiments, the amount of intersection is determined based on first and second portions generated without using algorithms to automatically detect and correct for bite penetration.

For example, FIG. 6 illustrates a 3D digital representation 600 of a patient's dental arches with bite penetration, in accordance with embodiments of the present technology. The 3D digital representation 600 includes an upper portion 602 corresponding to the upper dental arch and a lower portion 604 corresponding to the lower dental arch, the upper portion 602 and lower portion 604 being positioned in occlusion with each other. In the illustrated example, the incisors of the upper portion 602 intersect the incisors of the lower portion 604 (indicated by arrows in FIG. 6) which is not physically possible in the patient's actual occlusion. Accordingly, the presence of these intersections in the 3D digital representation 600 can indicate issues with scanning accuracy.

Referring again to FIG. 5, at block 508, the method 500 can continue with evaluating scanning accuracy based on the amount of intersection. In some embodiments, the scanning accuracy is considered poor if the amount of intersection exceeds a threshold value (e.g., greater than 50 μm, 100 μm, 200 μm, or 500 μm), while small amounts of intersection (e.g., less than 10 μm) may be considered to be within normal tolerances of the scanning process. Significant amounts of intersection between the first and second portions representing the upper and lower arches may indicate accuracy problems with the single jaw scans and/or the bite scan. For instance, the intersections may be caused by plane deviation errors in which the teeth within a single arch do not lie in a flat bite plane. In embodiments where a confocal scanner system is used, plane deviation errors may arise from changes in field curvature causing the focal plane of the scanner to deviate from a flat plane.

Although the method 500 of FIG. 5 is described with respect to evaluating scanning accuracy based on intersections between upper and dental arches, scanning accuracy can alternatively or additionally be evaluated based on intersections between other intraoral structures that are physically impossible or implausible in the patient's actual dentition, such as intersections between neighboring teeth.

FIG. 7 is a flow diagram illustrating a method 700 for evaluating scanning accuracy based on comparing different scan data, in accordance with embodiments of the present technology. The method 700 can be implemented with any of the scanner systems described herein, such as the system 100 of FIGS. 1A and 1B. In some embodiments, some or all of the processes of the method 700 are implemented as computer-readable instructions (e.g., program code) that are configured to be executed by one or more processors of a computing device (e.g., a client device, a server device, or suitable combinations thereof). For example, any of the processes of the method 700 can be performed by the first computing device 104 and/or the second computing device 108 of the system 100 of FIGS. 1A and 1B. The method 700 may be combined with any of the other methods described herein, such as the method 200 of FIG. 2, the method 300 of FIG. 3, and/or the method 500 of FIG. 5.

At block 702, the method 700 can include generating a first 3D digital representation from first scan data, and at block 704, the method 700 can include generating a second 3D digital representation from second scan data. The first and second scan data can be obtained during the same intraoral scanning session and/or can depict the same intraoral structure, such that the first and second 3D digital representations are expected to be the same or similar, absent scanning errors. However, the first and second scan data can include different scans, such that discrepancies between the first and second 3D digital representations may be used to detect scanning errors and/or identify the source(s) of scanning errors.

For example, FIG. 8A schematically illustrates scan data 802 divided into a set of first scans 804 and a set of second scans 806, in accordance with embodiments of the present technology. The first scans 804 can be registered and stitched to each other to generate a first 3D digital representation 808, and the second scans 806 can be registered and stitched to each other to generate a second 3D digital representation 810. In the illustrated embodiment, the first scans 804 are even-numbered scans and the second scans 806 are odd-numbered scans. Discrepancies between the first 3D digital representation 808 and the second 3D digital representation 810 can be indicative of excessive noise in the scanner system, for example.

Alternatively, in embodiments where the scanner includes a plurality of optical elements (e.g., a plurality of cameras and/or a plurality of structured light projectors), the first scans 804 can include scan data obtained using a first set of the optical elements (e.g., a first set of cameras and/or structured light projectors), and the second scans 806 can include scan data obtained using a second, different set of the optical elements (e.g., a second set of cameras and/or structured light projectors). In such embodiments, discrepancies between the first 3D digital representation 808 and the second 3D digital representation 810 can be indicative of hardware and/or calibration issues in the first set of optical elements, the second set of optical elements, or both.

As another example, FIG. 8B schematically illustrates scan data 812 including a selected subset of scans 814, in accordance with embodiments of the present technology. A first 3D digital representation 816 can be generated using the entire set of scan data 812, and a second 3D digital representation 818 can be generated using only the selected subset of scans 814. For instance, the subset of scans 814 can include only 95%, 90%, 85%, 80%, 75%, or 50% of the total scan data 812. The subset of scans 814 can be selected randomly or based on other criteria (e.g., dropping one scan per every X scans). Discrepancies between the first 3D digital representation 816 and the second 3D digital representation 818 can be indicative of excessive noise in the scanner system, for example.

Referring again to FIG. 7, at block 706, the method 700 can continue with comparing the first 3D digital representation to the second 3D digital representation. The comparison can include detecting a discrepancy between the first and second 3D digital representations. For instance, the geometry (e.g., shape, size) of the first and second 3D digital representations can be compared to each other to identify the extent and/or location of any discrepancies, e.g., by registering the first and second 3D digital representations to each other and then calculating the distances between the corresponding surfaces. Significant deviations between the first and second 3D digital representations may be indicative of scanning errors, as discussed above.

At block 708, the method 700 can include evaluating scanning accuracy based on the comparison. In some embodiments, the scanning accuracy is considered poor if there is a significant discrepancy between the first and second 3D digital representations, e.g., the average and/or maximum deviation exceeds a threshold value.

Although the method 700 of FIG. 7 is described with respect to evaluating scanning accuracy by comparing two 3D digital representations generated from two different sets of scan data, scanning accuracy can be evaluated by comparing any number of 3D digital representations generated from any number of different sets of scan data, such as three, four, five, or more different sets of scan data for generating three, four, five, or more 3D digital representations for comparison. The scan data for each 3D digital representation can be selected in any suitable manner, e.g., randomly, based on different sets of optical components, based on different numbers of scans, etc.

Optionally, the method 700 is performed to identify the source of poor scanning accuracy, after poor scanning accuracy has been detected using another method described herein. For instance, the method 300 of FIG. 3 and/or the method 500 of FIG. 5 can be performed first to detect poor scanning accuracy based on stitching errors and/or bite penetration. Subsequently, the method 700 can be performed to generate 3D digital representations using different sets of scan data to determine the source of the poor scanning accuracy. For instance, if the 3D digital representation generated using a particular camera deviates significantly from 3D digital representations generated using other cameras, the method 700 can identify that particular camera as being a potential cause of the accuracy issues.

### EXAMPLES

The following examples are included to further describe some aspects of the present technology, and should not be used to limit the scope of the technology.

Example 1. An intraoral scanning system comprising:
a scanner;
one or more processors; and
a memory operably coupled to the one or more processors and storing instructions that, when executed by the one or more processors, cause the system to perform operations comprising:
   receiving results of at least one intraoral scanning session, wherein the at least one intraoral scanning session comprises:
      obtaining scan data of an intraoral structure from the scanner, and
      combining the scan data to generate a 3D digital representation of the intraoral structure;
   evaluating scanning accuracy of the scanner based on the results of the at least one intraoral scanning session; and
   outputting an indication of the scanning accuracy.

Example 2. The system of Example 1, wherein the scanning accuracy is evaluated based on the results of a single intraoral scanning session.

Example 3. The system of Example 1, wherein the scanning accuracy is evaluated based on the results of a plurality of intraoral scanning sessions.

Example 4. The system of Example 3, wherein evaluating the scanning accuracy comprises:
generating an accuracy measurement for at least some of the intraoral scanning sessions, and
detecting a change in the scanning accuracy over the plurality of intraoral scanning sessions by comparing the accuracy measurements of the at least some of the intraoral scanning sessions.

Example 5. The system of Example 4, wherein the operations further comprise identifying a cause of the change in the scanning accuracy based on the accuracy measurements of the at least some of the intraoral scanning sessions.

Example 6. The system of any one of Examples 1 to 5, wherein the indication comprises a recommendation to take one or more of the following actions: repeat an intraoral scanning session with the scanner, recalibrate the scanner, repair the scanner, or replace the scanner.

Example 7. The system of any one of Examples 1 to 6, wherein the indication shows whether the scanning accuracy is below a predetermined threshold value.

Example 8. The system of any one of Examples 1 to 7, further comprising a display, wherein the indication of the scanning accuracy is output on the display.

Example 9. The system of any one of Examples 1 to 8, wherein the indication of the scanning accuracy is output to a remote computing device.

Example 10. The system of any one of Examples 1 to 9, wherein the scan data comprises a plurality of scans, and wherein combining the scan data comprises:
registering at least two scans to each other, and
stitching the at least two scans together to generate at least a portion of the 3D digital representation.

Example 11. The system of Example 10, wherein the operations further comprise determining whether a stitching error is present, wherein the scanning accuracy is evaluated based on presence of the stitching error.

Example 12. The system of Example 11, wherein:
the 3D digital representation comprises:
   a first portion generated from first scan data obtained while the scanner is moved over a location on the intraoral structure along a first direction, and
   a second portion generated from second scan data obtained while the scanner is moved over the location on the intraoral structure along a second direction, and
the presence of the stitching error is determined based on a discrepancy between the first and second portions of the 3D digital representation.

Example 13. The system of Example 12, wherein the discrepancy comprises a discontinuity between the first and second portions of the 3D digital representation.

Example 14. The system of any one of Examples 1 to 13, wherein:
the 3D digital representation comprises a first portion corresponding to an upper dental arch and a second portion corresponding to a lower dental arch, the first and second portions being positioned in occlusion with each other, and
the scanning accuracy is evaluated based on an amount of intersection between the first and second portions.

Example 15. The system of any one of Examples 1 to 14, wherein:
combining the scan data comprises:
   generating a first 3D digital representation of the intraoral structure from first scan data, and
   generating a second 3D digital representation of the intraoral structure from second scan data, and
the scanning accuracy is evaluated based on a comparison between the first 3D digital representation and the second 3D digital representation.

Example 16. The system of Example 15, wherein the first scan data comprises even-numbered scans and the second scan data comprises odd-numbered scans.

Example 17. The system of Example 15 or 16, wherein the first scan data comprises a different number of scans than the second scan data.

Example 18. The system of any one of Examples 15 to 17, wherein the scanner comprises a plurality of optical elements, the first scan data is obtained using a first set of the optical elements, and the second scan data is obtained using a second set of the optical elements.

Example 19. The system of Example 18, wherein the plurality of optical elements comprise a plurality of cameras.

Example 20. The system of Example 18 or 19, wherein the plurality of optical elements comprise a plurality of structured light projectors.

Example 21. The system of any one of Examples 1 to 20, wherein the operations further comprise obtaining additional data of the intraoral structure, wherein the scanning accuracy is evaluated based on a comparison of the 3D digital representation of the intraoral structure and the additional data.

Example 22. The system of Example 21, wherein the additional data comprises cone beam computed tomography (CBCT) data.

Example 23. A method comprising:
receiving results of at least one intraoral scanning session, wherein the at least one intraoral scanning session comprises:
   obtaining scan data of an intraoral structure from a scanner, and
   combining the scan data to generate a 3D digital representation of the intraoral structure;
evaluating scanning accuracy of the scanner based on the results of the at least one intraoral scanning session; and
outputting an indication of the scanning accuracy on a display.

Example 24. The method of Example 23, wherein the scanning accuracy is evaluated based on results of a single intraoral scanning session.

Example 25. The method of Example 23, wherein the scanning accuracy is evaluated based on results of a plurality of intraoral scanning sessions.

Example 26. The method of Example 25, wherein the plurality of intraoral scanning procedures are performed using the same scanner.

Example 27. The method of Example 25, wherein the plurality of intraoral scanning sessions are performed using different scanners.

Example 28. The method of any one of Examples 25 to 27, wherein the plurality of intraoral scanning sessions differ from each other with respect to one or more of the following: scanner type, scanning protocol, scanner manufacturing method, scanning calibration method, scanning software, user, scanner computer processor, scanner computer processor type, scanner sleeve, or scanner sleeve type.

Example 29. The method of any one of Examples 25 to 28, wherein evaluating the scanning accuracy comprises:
generating an accuracy measurement for at least some of the intraoral scanning sessions, and
detecting a change in the scanning accuracy over the plurality of intraoral scanning sessions by comparing the accuracy measurements of the at least some of the intraoral scanning sessions.

Example 30. The method of Example 29, further comprising identifying a cause of the change in the scanning accuracy based on the accuracy measurements of the at least some of the intraoral scanning sessions.

Example 31. The method of any one of Examples 23 to 30, wherein the indication comprises a recommendation to take one or more of the following actions: repeat an intraoral scanning session with the scanner, recalibrate the scanner, repair the scanner, or replace the scanner.

Example 32. The method of any one of Examples 23 to 31, wherein the indication shows whether the scanning accuracy is below a predetermined threshold value.

Example 33. The method of any one of Examples 23 to 32, wherein the at least one intraoral scanning session is performed by a first computing device, and the receiving, evaluating, and outputting processes are performed by the first computing device.

Example 34. The method of any one of Examples 23 to 33, wherein the at least one intraoral scanning session is performed by a first computing device, and the receiving, evaluating, and outputting processes are performed by a second computing device different than the first computing device.

Example 35. The method of any one of Examples 23 to 34, wherein the scan data comprises a plurality of scans, and wherein combining the scan data comprises:
registering at least two scans to each other, and
stitching the at least two scans together to generate at least a portion of the 3D digital representation.

Example 36. The method of Example 35, further comprising wherein the operations further comprise determining whether a stitching error is present, wherein the scanning accuracy is evaluated based on presence of the stitching error.

Example 37. The method of Example 36, wherein:
the 3D digital representation comprises:
   a first portion generated from first scan data obtained while the scanner is moved over a location on the intraoral structure along a first direction, and
   a second portion generated from second scan data obtained while the scanner is moved over the location on the intraoral structure along a second direction, and
the presence of the stitching error is determined based on a discrepancy between the first and second portions of the 3D digital representation.

Example 38. The method of Example 37, wherein the discrepancy comprises a discontinuity between the first and second portions of the 3D digital representation.

Example 39. The method of any one of Examples 23 to 38, wherein:
the 3D digital representation comprises a first portion corresponding to an upper dental arch and a second portion corresponding to a lower dental arch, the first and second portions being positioned in occlusion with each other, and
the scanning accuracy is evaluated based on an amount of intersection between the first and second portions.

Example 40. The method of any one of Examples 23 to 39, wherein:
combining the scan data comprises:
   generating a first 3D digital representation of the intraoral structure from first scan data, and
   generating a second 3D digital representation of the intraoral structure from second scan data, and
the scanning accuracy is evaluated based on a comparison between the first 3D digital representation and the second 3D digital representation.

Example 41. The method of Example 40, wherein the first scan data comprises even-numbered scans and the second scan data comprises odd-numbered scans.

Example 42. The method of Example 40 or 41, wherein the first can data comprises a different number of scans than the second scan data.

Example 43. The method of any one of Examples 40 to 42, wherein the scanner comprises a plurality of optical elements, the first scan data is obtained using a first set of the optical elements, and the second scan data is obtained using a second set of the optical elements.

Example 44. The method of Example 43, wherein the plurality of optical elements comprise a plurality of cameras.

Example 45. The method of Example 43 or 44, wherein the plurality of optical elements comprise a plurality of structured light projectors.

Example 46. The method of any one of Examples 23 to 45, further comprising obtaining additional data of the intraoral structure, wherein the scanning accuracy is evaluated based on a comparison of the 3D digital representation of the intraoral structure and the additional data.

Example 47. The method of Example 46, wherein the additional data comprises cone beam computed tomography (CBCT) data.

Example 48. A non-transitory computer-readable storage medium comprising instructions that, when executed by one or more processors of a computing system, cause the computing system to perform operations comprising the method of any one of Examples 23 to 47.

Example 49. A system for monitoring scanning accuracy, the system comprising:
one or more processors; and
a memory operably coupled to the one or more processors and storing instructions that, when executed by the one or more processors, cause the system to perform operations comprising:
   receiving results of a plurality of intraoral scanning sessions, wherein each intraoral scanning session is performed using a respective scanner, and wherein each intraoral scanning session comprises:
      obtaining scan data of an intraoral structure from the respective scanner, and
      combining the scan data to generate a 3D digital representation of the intraoral structure;
   evaluating scanning accuracy of the plurality of intraoral scanning sessions, based on the received results; and
   outputting, on a display, an indication of the scanning accuracy.

Example 50. The system of Example 49, wherein the plurality of intraoral scanning sessions differ from each other with respect to one or more of the following scanning parameters: scanner type, scanning protocol, scanner manufacturing method, scanning calibration method, scanning software, or user.

Example 51. The system of Example 50, wherein the operations further comprise:
comparing the scanning accuracy of the plurality of intraoral scanning sessions, and
identifying a scanning parameter associated with poor scanning accuracy, based on the comparison.

Example 52. The system of Example 51, wherein the indication comprises the identified scanning parameter.

Example 53. The system of Example 50 or 51, wherein the indication comprises a recommendation for a corrective action to address the poor scanning accuracy.

Example 54. A system for monitoring scanning accuracy, the system comprising:
one or more processors; and
a memory operably coupled to the one or more processors and storing instructions that, when executed by the one or more processors, cause the system to perform operations comprising:
   receiving results of a plurality of intraoral scanning sessions performed using a scanner, wherein each intraoral scanning session is performed at a different time, and wherein each intraoral scanning session comprises:
      obtaining scan data of an intraoral structure from the scanner, and
      combining the scan data to generate a 3D digital representation of the intraoral structure;
   evaluating scanning accuracy of the scanner over time, based on the received results; and
   outputting, on a display, an indication of the scanning accuracy.

Example 55. The system of Example 54, wherein evaluating the scanning accuracy comprises:
generating an accuracy measurement for each of the plurality of intraoral scanning sessions, and
detecting a change in the scanning accuracy over time by comparing the accuracy measurements of the plurality of intraoral scanning sessions.

Example 56. The system of Example 55, wherein the operations further comprise identifying a cause of the change in the scanning accuracy based on the accuracy measurements of the plurality of intraoral scanning sessions.

Example 57. The system of Example 56, wherein the identified cause comprises one or more of the following: scanner hardware malfunction, scanner software malfunction, an issue with a computing device associated with the scanner, calibration drift, noise, or user error.

Example 58. The system of Example 56 or 57, wherein the indication comprises the identified cause and a recommendation for a corrective action to address the identified cause.

### CONCLUSION

Although many of the embodiments are described above with respect to systems, devices, and methods for intraoral scanning, the technology is applicable to other applications and/or other approaches, such as scanning of other anatomical locations or other types of objects. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-8B.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. An intraoral scanning system comprising:
a scanner;
one or more processors; and
a memory operably coupled to the one or more processors and storing instructions that, when executed by the one or more processors, cause the intraoral scanning system to perform operations comprising:
receiving results of at least one intraoral scanning session, wherein the at least one intraoral scanning session comprises:
obtaining scan data of an intraoral structure from the scanner, and
combining the scan data to generate a 3D digital representation of the intraoral structure;
evaluating scanning accuracy of the scanner based on the results of the at least one intraoral scanning session; and
outputting, on a display, an indication of the scanning accuracy.

2. The system of claim 1, wherein the scanning accuracy is evaluated based on the results of a single intraoral scanning session.

3. The system of claim 1, wherein the scanning accuracy is evaluated based on the results of a plurality of intraoral scanning sessions.

4. The system of claim 3, wherein evaluating the scanning accuracy comprises:
generating an accuracy measurement for at least some of the intraoral scanning sessions, and
detecting a change in the scanning accuracy over the plurality of intraoral scanning sessions by comparing the accuracy measurements of the at least some of the intraoral scanning sessions.

5. The system of claim 4, wherein the operations further comprise identifying a cause of the change in the scanning accuracy based on the accuracy measurements of the at least some of the intraoral scanning sessions.

6. The system of claim 1, wherein the indication comprises a recommendation to take one or more of the following actions: repeat an intraoral scanning session with the scanner, recalibrate the scanner, repair the scanner, or replace the scanner.

7. The system of claim 1, wherein the indication shows whether the scanning accuracy is below a predetermined threshold value.

8. The system of claim 1, wherein:
the 3D digital representation comprises:
a first portion generated from first scan data obtained while the scanner is moved over a location on the intraoral structure along a first direction, and
a second portion generated from second scan data obtained while the scanner is moved over the location on the intraoral structure along a second direction, and
the scanning accuracy is evaluated based on whether a stitching error is present, wherein the presence of the stitching error is determined based on a discrepancy between the first and second portions of the 3D digital representation.

9. The system of claim 1, wherein:
the 3D digital representation comprises a first portion corresponding to an upper dental arch and a second portion corresponding to a lower dental arch, the first and second portions being positioned in occlusion with each other, and
the scanning accuracy is evaluated based on an amount of intersection between the first and second portions.

10. The system of claim 1, wherein:
combining the scan data comprises:
generating a first 3D digital representation of the intraoral structure from first scan data, and
generating a second 3D digital representation of the intraoral structure from second scan data, and
the scanning accuracy is evaluated based on a comparison between the first 3D digital representation and the second 3D digital representation.

11. A system for monitoring scanning accuracy, the system comprising:
one or more processors; and
a memory operably coupled to the one or more processors and storing instructions that, when executed by the one or more processors, cause the system to perform operations comprising:
receiving results of a plurality of intraoral scanning sessions, wherein each intraoral scanning session is performed using a respective scanner, and wherein each intraoral scanning session comprises:
obtaining scan data of an intraoral structure from the respective scanner, and
combining the scan data to generate a 3D digital representation of the intraoral structure;
evaluating scanning accuracy of the plurality of intraoral scanning sessions, based on the received results; and
outputting, on a display, an indication of the scanning accuracy.

12. The system of claim 11, wherein the plurality of intraoral scanning sessions differ from each other with respect to one or more of the following scanning parameters: scanner type, scanning protocol, scanner manufacturing method, scanning calibration method, scanning software, or user.

13. The system of claim 12, wherein the operations further comprise:
comparing the scanning accuracy of the plurality of intraoral scanning sessions, and
identifying a scanning parameter associated with poor scanning accuracy, based on the comparison.

14. The system of claim 13, wherein the indication comprises the identified scanning parameter.

15. The system of claim 13, wherein the indication comprises a recommendation for a corrective action to address the poor scanning accuracy.

16. A system for monitoring scanning accuracy, the system comprising:
one or more processors; and
a memory operably coupled to the one or more processors and storing instructions that, when executed by the one or more processors, cause the system to perform operations comprising:
receiving results of a plurality of intraoral scanning sessions performed using a scanner, wherein each intraoral scanning session is performed at a different time, and wherein each intraoral scanning session comprises:
obtaining scan data of an intraoral structure from the scanner, and
combining the scan data to generate a 3D digital representation of the intraoral structure;
evaluating scanning accuracy of the scanner over time, based on the received results; and
outputting, on a display, an indication of the scanning accuracy.

17. The system of claim 16, wherein evaluating the scanning accuracy comprises:
generating an accuracy measurement for each of the plurality of intraoral scanning sessions, and
detecting a change in the scanning accuracy over time by comparing the accuracy measurements of the plurality of intraoral scanning sessions.

18. The system of claim 17, wherein the operations further comprise identifying a cause of the change in the scanning accuracy based on the accuracy measurements of the plurality of intraoral scanning sessions.

19. The system of claim 18, wherein the identified cause comprises one or more of the following: scanner hardware malfunction, scanner software malfunction, an issue with a computing device associated with the scanner, calibration drift, noise, or user error.

20. The system of claim 18, wherein the indication comprises the identified cause and a recommendation for a corrective action to address the identified cause.
